(19) United States
(12) Patent Application Publication
(10) Pub. No.: US 2024/0148993 A1
(43) Pub. Date: May 9, 2024

(54) PATIENT-VENTILATOR ASYNCHRONY DASHBOARD

(71) Applicant: Covidien LP, Mansfield, MA (US)

(72) Inventors: Pradnya S. Chaphekar, San Diego, CA (US); Cynthia A. Miller, Laguna Hills, CA (US); Gary S. Milne, Louisville, CO (US); Michal Marach, Erie, CO (US)

(73) Assignee: Covidien LP, Mansfield, MA (US)

(21) Appl. No.: 18/497,051

(22) Filed: Oct. 30, 2023

Related U.S. Application Data
(60) Provisional application No. 63/423,083, filed on Nov. 7, 2022.
(60) Provisional application No. 63/498,936, filed on Apr. 28, 2023.

(51) Int. Cl. A61M 16/00

(52) U.S. Cl. CPC A61M 16/0051; A61M 16/024; A61M 2205/502; A61M 2205/583; A61M 2230/40

(57) ABSTRACT

The present disclosure relates generally to medical ventilators, and more particularly to systems and methods for detecting and quantifying patient-ventilator asynchrony. In an example, a processor receives ventilation data from a medical ventilator and outputs a data dashboard on a display screen. The data dashboard displays a detection of first and second different types of patient-ventilator asynchrony events in the ventilation data. The data dashboard also includes time-scaled asynchrony metrics organized into tiles for each of the first and second asynchrony events.

### CROSS-REFERENCE TO RELATED APPLICATIONS

This application claims the benefit of U.S. Provisional Application No. 63/423,083 filed Nov. 7, 2022, and U.S. Provisional Application No. 63/498,936 filed Apr. 28, 2023, which applications are incorporated herein by reference in their entireties. To the extent appropriate a claim of priority is made to both applications.

### BACKGROUND

The present disclosure relates generally to medical ventilators and particularly to systems and methods for detecting and quantifying patient-ventilator asynchrony.

Mechanical ventilators provide breathing gases to patients in hospitals, nursing facilities, surgery centers, and other clinical environments (including home care). These ventilators deliver pressurized gases to a patient through a breathing hose (often called a circuit). At the patient, the breathing circuit connects to a patient interface such as a face mask, nasal mask, nasal cannula, endotracheal tube, or tracheostomy tube. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, combination ventilation modes, and patient directed ventilation modes.

In some instances, the breathing gases delivered to the patient may not be synchronous with the patient's own breathing efforts. The present disclosure is directed at improvements in this field.

### BRIEF DESCRIPTION OF THE DRAWINGS

Advantages of the disclosed techniques may become apparent upon reading the following detailed description and upon reference to the drawings in which:

FIG. 1 is a perspective view of a medical ventilator according to an embodiment of the present disclosure.

FIG. 2A depicts flow and pressure waveforms during a missed trigger event, according to an embodiment of the present disclosure.

FIG. 2B depicts flow and pressure waveforms during a double trigger event, according to an embodiment of the present disclosure.

FIG. 3A is a view of an asynchrony dashboard over a first time selection, according to an embodiment of the present disclosure.

FIG. 3B is a view of an asynchrony dashboard over a second time selection, according to an embodiment of the present disclosure.

FIG. 4A depicts a view of another asynchrony dashboard, according to an embodiment of the present disclosure.

FIG. 4B depicts a plot for identifying cluster-based asynchrony events, according to an embodiment of the present disclosure.

FIG. 5A is a view of an asynchrony clock, according to an embodiment of the present disclosure.

FIG. 5B is a view of another asynchrony clock, according to an embodiment of the present disclosure.

FIG. 5C is a view of another asynchrony clock, according to an embodiment of the present disclosure.

FIG. 6 is a view of an asynchrony clock, dashboard according to an embodiment of the present disclosure.

FIG. 7 depicts an example method for generating an asynchrony dashboard, according to an embodiment of the present disclosure.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

The present disclosure relates generally to medical ventilators, and more particularly to systems and methods for detecting and quantifying patient-ventilator asynchrony. In an aspect, the technology relates to a system for generating a patient-ventilator asynchrony dashboard. The system includes a processor receiving ventilation data from a medical ventilator and outputting a data dashboard on a display screen. The data dashboard displays a detection of first and second different types of patient-ventilator asynchrony events in the ventilation data. The data dashboard includes time-scaled asynchrony metrics organized into tiles for each of the first and second asynchrony events.

In an example, the first type of patient-ventilator asynchrony events includes ineffective efforts. In a further example, the second type of patient-ventilator asynchrony events includes double triggers. In another example, at least one of the tiles indicates a number of minutes out a set time period that the first type of patient-ventilator asynchrony events exceeded a threshold. In still another example, at least one of the tiles indicates percentage of time out a set time period that the first type of patient-ventilator asynchrony events exceeded a threshold. In yet another example, the data dashboard further comprises an asynchrony clock including at least one indicator representing an asynchrony. In still yet another example, the at least one indicator indicates a time, duration, and severity of the asynchrony.

In another aspect, the technology relates to a ventilator-implemented method for generating an asynchrony dashboard. The method includes receiving ventilation data during ventilation of a patient; based on the ventilation data, identifying asynchrony occurrences; generating an asynchrony signal based on the identified asynchrony occurrences, wherein the asynchrony signal represents a number of asynchrony occurrences per time frame; based on the asynchrony signal exceeding a threshold, detecting an asynchrony event, the asynchrony event having a duration and magnitude; and generating an asynchrony dashboard including at least one of tile or asynchrony clock including an indicator based on the detected asynchrony event, wherein the indicator indicates at least one of the duration or the magnitude of the detected asynchrony event.

In an example, the asynchrony signal is an ineffective effort signal. In another example, the asynchrony dashboard includes an asynchrony score based on at least one of duration or the magnitude of the asynchrony event. In yet another example, wherein the asynchrony score is based on a power of the asynchrony event. In still another example, the power indicates a number of asynchrony occurrences that occurred during the asynchrony event. In still yet another example, the dashboard includes the asynchrony clock. In a further example, the indicator indicates the magnitude, duration, and time of the asynchrony event. In another example, the asynchrony clock further includes at least one additional indicator corresponding to a manually entered event.

### DETAILED DESCRIPTION OF SPECIFIC EMBODIMENTS

The present disclosure includes systems and methods for detecting and quantifying patient-ventilator asynchrony in medical ventilation.

Patient-ventilator asynchrony occurs when the initiation and/or termination of mechanical breath from the ventilator is not in time agreement with the initiation or termination of the patient's own efforts to breathe (the patient's neural inspiration). As used herein, patient effort means a patient's spontaneous attempt to initiate an inhalation or an exhalation (to change the phase of the respiratory cycle from inspiration to exhalation or from exhalation to inspiration). Patient-ventilator asynchrony can be uncomfortable for the patient and may have an adverse impact on patient outcomes, including a higher or wasted work of breathing; diaphragm injury; patient discomfort; alveolar overdistention and lung injury; increased need of sedation; prolonged mechanical ventilation; longer hospital stays; and possibly higher mortality.

There are several types of patient-ventilator asynchrony, including but not limited to ineffective effort, double triggering, premature cycling, and delayed cycling. In addition, auto-positive end-expiratory pressure (PEEP) is a condition that commonly precedes or follows asynchrony, and thus the detection of auto-PEEP is included as a condition likely to lead to or indicate asynchrony. Each of the patient asynchrony types results from the ventilator triggering and/or cycling at an improper time that is out of synchronization with the patient's own breathing efforts.

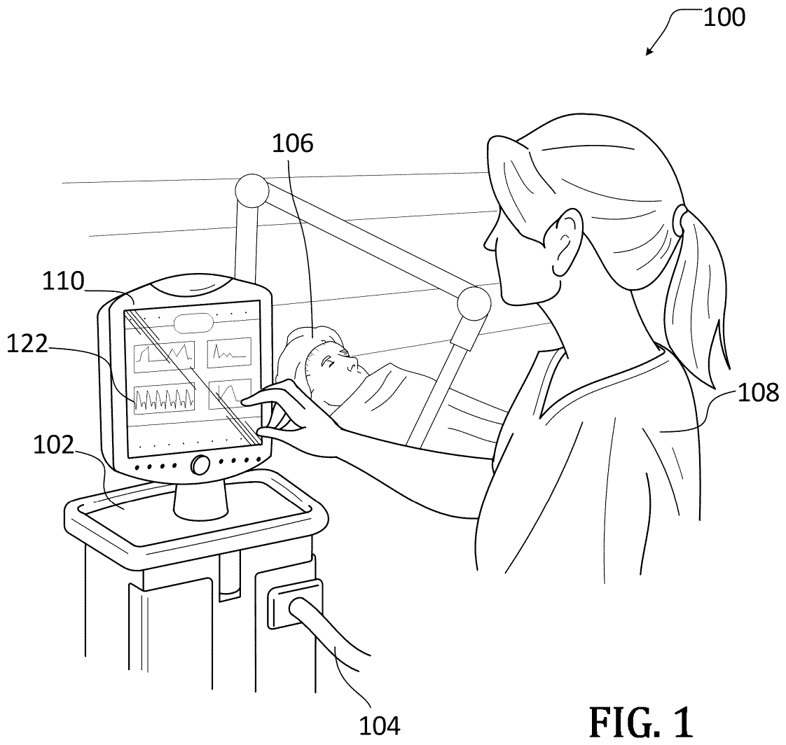

FIG. 1 shows a medical ventilator 100 according to an embodiment. The ventilator includes a breath delivery unit 102 that provides breathing gases to a patient 106 through a breathing circuit 104. A clinician 108 can monitor and adjust settings on the ventilator through a graphical user interface (GUI) 122 displayed on a display screen 110. The ventilator 100 includes sensors (such as flow, pressure, gas composition, and other sensors) that measure, derive, or calculate data regarding the breath delivery. During ventilation of a patient, the ventilator 100 generates a large amount of ventilation data. Asynchrony detection algorithms are used to monitor ventilation data for asynchrony events. Asynchrony detection includes analyzing ventilator waveforms, data, and/or other calculated values to detect asynchrony, including detecting the occurrence, frequency, timing, duration, severity, and/or type of asynchrony. As described further below, an asynchrony dashboard with asynchrony detection data is displayed on the ventilator screen 110 and/or on other remote screens and displays to assist medical caregivers in improving synchrony between the patient and the ventilator.

Three types of asynchronies (or conditions leading to asynchrony, in the case of auto-PEEP) are described in further detail below—ineffective effort, double trigger, and auto-PEEP. While these three asynchrony events are used as examples in the figures, it should be understood that the asynchrony dashboards of the present disclosure may include these and/or other types of asynchronies, including auto-triggering, premature cycling, delayed cycling, or others.

Ineffective Effort

An ineffective effort (also called ineffective trigger, missed trigger, or missed breath) is a patient inspiratory effort that does not result in the delivery of a breath by the ventilator. This missed breath occurs when the ventilator does not detect the inspiratory effort by the patient and, therefore, the ventilator does not deliver a breath in response to the patient's inspiratory effort.

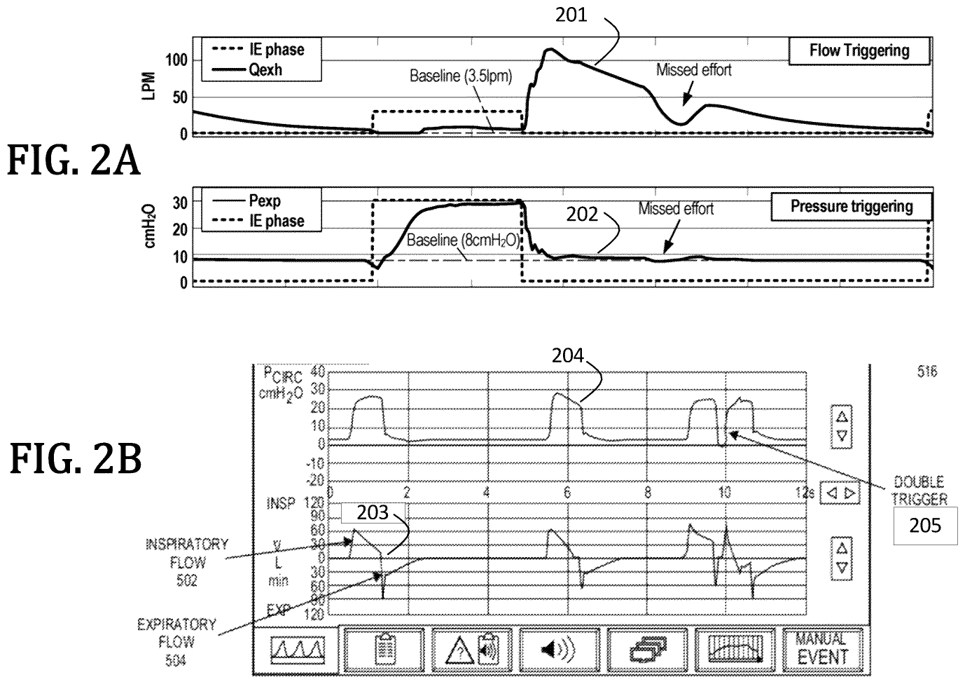

An example of an ineffective effort is shown in FIG. 2A, which shows flow and pressure waveforms during ventilation of a patient. The upper graph of shows an exhalation flow waveform 201 (Qexh) (dark solid line) plotted as LPM (liters per minute) over time. The graph also shows the phase of ventilation (IE phase) (dotted line raised during inhalation and lowered during exhalation). During exhalation, the flow waveform 201 drops as the rate of exhalation flow decreases. In a flow triggering mode, the ventilator is programmed to end exhalation and trigger a new inspiration when this flow waveform crosses a baseline by a set amount (e.g., a sensitivity setting). However, in the example shown in FIG. 2A, the patient makes an inhalation effort before the end of the exhalation phase. This effort is visible where the flow waveform 201 dips downward, as the patient attempts to draw air inward (and thus exhalation flow decreases). In this example, because the flow waveform does not pass below the baseline threshold, the ventilator does not trigger a new breath, and the result is a missed trigger. The patient demanded a new breath, but the ventilator did not trigger inspiration. The missed patient effort is also visible in the lower graph, which shows a pressure waveform 202 (Pexp) (plotted in units of cmH₂O). In the waveform 202, the patient's inspiratory effort was missed because the expiratory pressure (Pexp) did not drop below the trigger baseline.

In an embodiment, the ventilator 100 (or remote processor) runs a background/analytic trigger detection module that detects patient efforts to trigger a new breath. Any detected effort that does not correlate with a delivered breath is a missed breath or missed trigger. That patient effort was an ineffective effort. Further discussion of missed breath analysis is shown in U.S. Pat. No. 10,362,967, titled Systems and Methods for Missed Breath Detection and Indication, and U.S. application Ser. No. 17/890,957, titled Automatic Synchronization for Medical Ventilation, the contents of both of which are incorporated herein by reference in their entireties.

Double Trigger

Double triggering refers to the occurrence of two consecutive inspirations with one patient inspiratory effort. In this scenario, a ventilator delivers two breaths in response to what is, in fact, a single patient effort. Double triggering generally occurs when a patient's respiratory drive is high, ventilator support is insufficient compared to patient effort, or the patient's neural inspiratory time is longer than ventilator delivered inspiratory time. The set inspiratory time may be too short (compared to neural time), or the expiratory trigger threshold (e.g., expiratory sensitivity setting) may be too high. Double triggering may provoke high tidal volume (up to twice the set value) putting the patient at risk of ventilator-induced lung injury and ventilator-induced diaphragmatic dysfunction.

During a double-triggered event, the patient's respiratory effort continues even during the exhalation phase. The exhalation phase is generally short and the expired volume is much smaller compared to the inspired volume since the patient's respiratory effort continues. Double triggering can be detected based on these characteristics. Further discussion of double trigger detection is shown in U.S. Pat. No. 8,757,153, titled Ventilator-Initiated Prompt Regarding Detection of Double Triggering During Ventilation, the contents of which are incorporated herein by reference in its entirety.

An example of a double trigger asynchrony event is shown in FIG. 2B, which shows flow 203 and pressure 204 waveforms during ventilation of a patient. Around time 10 seconds in the graph, two breaths are delivered with a very short expiratory phase between them. This double breath is labeled on the graph as a double trigger event 205.

Auto-PEEP

Auto-PEEP (also called intrinsic PEEP, air trapping, gas trapping) is another type of asynchrony event.

The pressure at the end of exhalation, from which a ventilator initiates inspiration, is termed the end-expiratory pressure (EEP) and, when EEP is positive, it is termed positive end-expiratory pressure (PEEP). PEEP may be prescribed to support oxygenation and prevent alveolar collapse at the end of expiration. Auto-PEEP means that more PEEP is left in the lungs at the end of exhalation than the PEEP amount set to be delivered by the ventilator.

Auto-PEEP may result when the lungs are not sufficiently emptied during expiration before inspiration is initiated. The exhalation phase ends before lung pressure is able to equilibrate with the ventilator breathing circuit pressure. For non-triggering patients in mandatory breath modes, auto-PEEP can happen when the respiratory rate is set too high, inspiratory pressure or tidal volume is too high, inspiratory time is too long, and/or expiratory time is too short. For triggering patients in mandatory or spontaneous breath modes, auto-PEEP may result when inspiration is too long, resulting in a short exhalation time before the next inspiration is triggered by the patient. When incomplete exhalation occurs, gases may be trapped in the lungs. With certain breath types, each breath may result in additional gases being trapped. Auto-PEEP has been linked to barotrauma, an increase in the work of breathing (WOB), missed triggers, and other problems.

If end-expiratory flow (EEF) exceeds a threshold, the pressure gradient between the patient's lungs and the ambient surface pressure (breathing circuit pressure) has likely not reached zero. As such, it is likely that gases have not been completely exhaled, and auto-PEEP can be detected. Auto-PEEP detection is described in more detail in U.S. Pat. No. 8,607,791, titled Ventilator-Initiated Prompt Regarding Auto-PEEP Detection During Pressure Ventilation, the contents of which are incorporated herein by reference in its entirety.

Asynchrony Dashboard

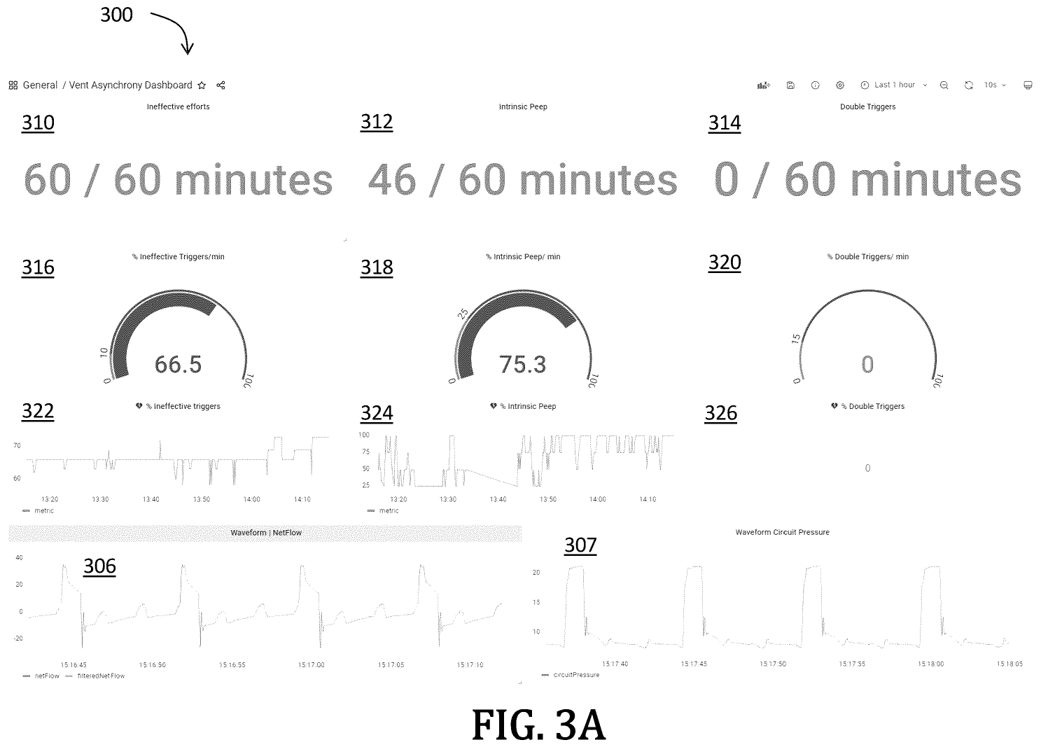

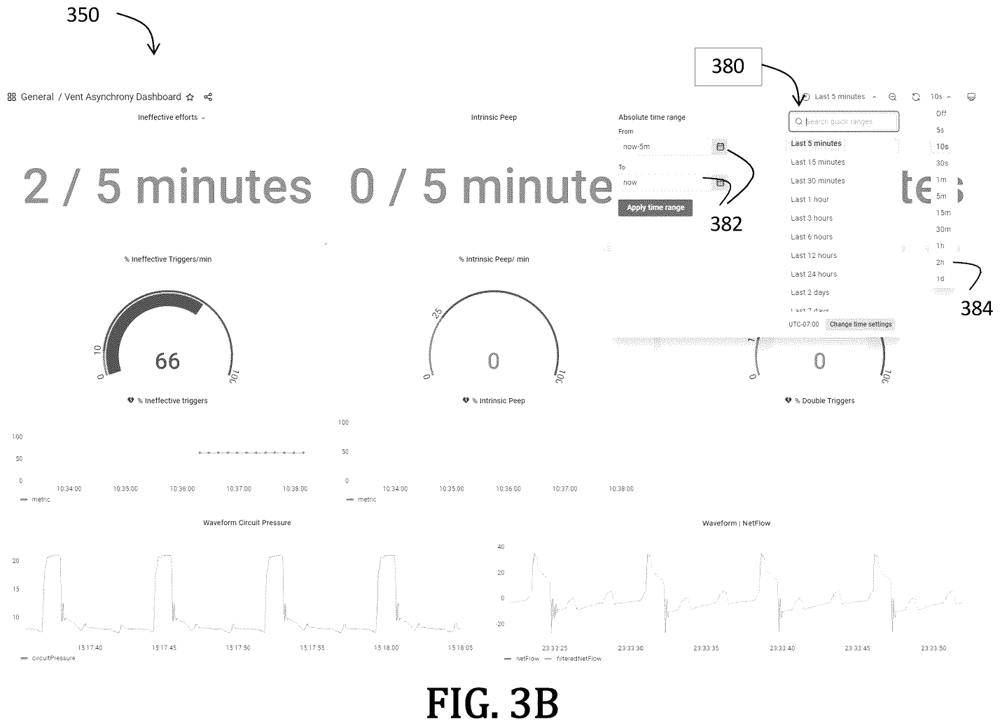

According to an embodiment, an asynchrony dashboard is generated and displayed to detect and quantify asynchrony events in ventilation data of a patient. An example asynchrony dashboard 300 is shown in FIG. 3A. The example asynchrony dashboard 300 identifies three types of patient-ventilator asynchrony events and displays an analysis of these events in dashboard tiles. Ineffective efforts are quantified in the tiles on the left, auto-PEEP (intrinsic PEEP) in the middle, and double triggers on the right. The data is presented over a time duration, which may be configurable. For instance, a user selection may be received that configures the time duration over which the data is to be presented. In the example depicted, and as discussed further below with reference to FIG. 3B, a selectable user interface element in the top-right side of the dashboard 300 may be selected to cause the display of a configuration menu. In FIG. 3A, the time duration selected is the most recent 60 minutes. At the bottom of the dashboard are two plots 306 and 307 that continuously update to show rolling (substantially real-time) pressure and flow waveforms from the ventilator.

The dashboard 300 presents asynchrony information on a time-scaled platform, in order to give the clinician an understanding of the extent and severity of the asynchrony problem over specific time durations—including the type of asynchrony experienced, the frequency of the asynchrony, and the severity of the asynchrony. The dashboard 300 collects data into time-based metrics such as consistency (for example, the number of minutes with asynchrony over the time duration) and rate (for example, the rate of asynchronous breaths per minute). This creative view of asynchrony data enables a clinician to quickly understand the existence and severity of an asynchrony problem for a particular patient, and then take necessary steps to adjust ventilation strategy for the patient.

The dashboard 300 detects and quantifies asynchrony events experienced by the patient over the selected time frame and presents this information in a way that can be quickly understood by a medical professional. The dashboard does more than just detect asynchrony events; asynchrony detection is one of several inputs into the dashboard. The dashboard output is a comprehensive asynchrony platform organized into a tile-based dashboard view of multiple different types of asynchrony events over different time periods.

In FIG. 3A, the dashboard 300 interprets and displays asynchrony event data over a selected period of time, which in the figure is the preceding 60 minutes (see top right indication of “Last 1 hour”). In the top row of tiles, the dashboard shows the number of minutes (during the selected time frame) in which the asynchrony event was detected. In tile 310, the dashboard identifies that in all 60 of the past 60 minutes, an ineffective effort event was detected. In tile 312, the dashboard identifies that in 46 of the past 60 minutes, an auto-PEEP event was detected. In tile 314, the dashboard identifies that no double triggers took place in the last 60 minutes (showing zero minutes out of 60). This top row quantifies how consistently the asynchrony event is occurring during the selected time frame. By toggling between time durations, a user can quickly identify when asynchrony events are occurring.

The second row of tiles quantifies the severity of the detected asynchrony events, by showing the rate of occurrence within each minute. In this example, the rate is shown as the percentage of breaths in which the asynchrony was present (e.g., the number is the average over the selected time duration). For example, tile 316 shows that an ineffective effort was detected in connection with 66.5% of breaths during the past hour. Tile 318 shows that auto-PEEP was detected in connection with 75.3% of breaths during the past hour, and tile 320 shows zero double triggers.

The second row also shows the threshold percentage defining the detection of the asynchrony. For example, tile 316 shows a threshold value of 10%. This means that for this dashboard analysis, an ineffective effort asynchrony is detected in a particular minute if at least 10% of breaths during that minute are associated with an ineffective effort. If ineffective efforts occur less frequently than this threshold during a one-minute duration, then that minute is counted as having zero asynchrony events. This threshold helps to differentiate isolated asynchrony events from more concerning recurring events. Tile 318 shows that auto-PEEP is detected in a particular minute if 25% or more of breaths within that minute are associated with an auto-PEEP event. Tile 320 shows that double trigger asynchrony is detected in a minute of 15% or more of breaths within that minute are associated with a double-trigger event.

The dashboard 300 shows at a glance that ineffective efforts far exceeded the 10% threshold (tile 316) in 60 out of the past 60 minutes (tile 310). The dashboard 300 also shows that auto-PEEP far exceeded the 25% threshold (tile 318) in 46 out of the past 60 minutes (tile 312). The dashboard 300 further shows that double triggering is not taking place (tiles 320 and 314). These tiles make this analysis easy to understand in a single dashboard view.

Tiles 322, 324, and 326 show the percentages over the full selected time period (60 minutes). Tile 322 shows that the percentage per minute of breaths with ineffective efforts (over the past 60 minutes) moved up and down between about 60% and 70% (averaging 66.5%). Tile 324 shows that the percentage per minute of breaths with auto-PEEP moved up and down between about 25% and 100% (averaging 75.3%). Such trends of the percentage of asynchrony events may provide insights to allow for readily determining changes in patient condition and/or breath delivery settings that may be contributing to an increase or decrease in asynchrony events.

At the bottom of the dashboard, real-time plots 306, 307 show a rolling 30-second window of a pressure and flow data from the ventilator. In the example depicted, these plots 306, 307 have a separate time frame than the tiles described above. For instance, the time frame for the real-time plots 306, 307 may be shorter than the time frame selected for the other asynchrony tiles. The shorter time frame allows for a clearer depiction of the pressure and flow waveforms for recent breaths. The plots 306, 307 show real-time data, such as the most recent 10, 15, 20, 30, or 60 seconds of data, on a rolling basis. In examples, this rolling window of time is not the same as the retrospective time duration that is selected by the user for the tiles. In some examples, the real-time plots 306, 307 may include waveforms for ventilation parameters other than pressure and flow, such as signals that are derived from and/or based on pressure and/or flow signals (e.g., volume). The particular ventilation parameters that are used for the real-time plots 306, 307 may be selectable or configurable by the user through the configuration menus discussed herein.

FIG. 3B shows another dashboard 350 showing time-based asynchrony metrics over the past 5 minutes. This view of the dashboard 350 also shows one example of a configuration menu in the form of a drop-down menu 380 from which the user can select a time duration. The drop-down menu 380 may be activated by selecting a corresponding activation user interface element from within the dashboard 300. The menu 380 presents a variety of quick ranges, which are predefined ranges of time that can be selected. The quick-range options shown in the menu 380 range from the last 5 minutes to the last 7 days. A custom range can also be entered. For instance, fields 382 may be provided in which a particular “from” date/time (e.g., a time in the past) and a “to” time may be entered (e.g., the current time or a time after the “from” date/time).

A refresh-rate menu 384 also enables the user to adjust how frequently the entire dashboard updates with refreshed data. The refresh-rate menu 384 may also be accessed by selecting a corresponding activation user interface element displayed within the dashboard. While the refresh-rate menu 384 is depicted as being separate from the configuration menu 380, in other examples the menus 380, 384 may be combined. For instance, the options presented in the refresh-rate menu 384 may be combined with the configuration menu 380.

While the menus 380, 384 are depicted as being drop-down menus that are activated through the selection of a user interface element, the menus may be presented in different forms in other examples. For instance, the menu may be displayed as a popup menu, a pane menu, or other types of menus. In some examples, the time frames in menus 380, 384 can be adjusted by gestures on the dashboard 350, such as pinching fingers to zoom in (shorten) or spreading fingers to expand (lengthen) the time duration, or other gestures.

The example dashboard 350 shows the asynchrony data for the past 5 minutes, which is the time duration set in the configuration menu 380. The data in dashboard 350 shows that ineffective efforts took place at a high rate (66% per minute) but only in 2 of the past 5 minutes, and auto-PEEP and double triggers were not present.

The tiles, numbers, waveforms, graphs, and other metrics in the dashboards 300, 350 can be color-coded to flag tiles where asynchrony events are detected or asynchrony data has changed. For instance, tiles including metrics that exceed a particular threshold, may be displayed in a particular color (or format) or having a border of particular color or format). Multiple thresholds may be implemented such that multiple different colors may be used (e.g., green, yellow, red). The particular formats and thresholds may also be configurable by a user though one or more configuration menus that may be presented via interactions with the dashboard 350. The thresholds may also be tile and/or asynchrony-type dependent. For instance, different thresholds may be implemented for each tile and/or each different asynchrony type.

In some examples, additional data can be accessed by selecting individual tiles in the dashboard (such as by clicking, tapping, and/or long-pressing a tile). This additional data may include ventilator settings and settings changes, alarm conditions, logs, patient data, and/or other ventilatory data over the selected time period or longer time periods for the patient. For example, referring back to FIG. 3A and dashboard 300, when the user taps on a tile in the Ineffective Efforts panel (tile 310, 316, or 322), the dashboard 300 may open another window (or replace or augment the tile) with additional data relevant to ineffective efforts, over the selected time period. This additional data includes ventilator settings data and patient data. In some examples, when the user taps on a tile in the Double Triggers panel (tile 314, 320, or 326), the dashboard 300 opens another window (or replaces or augments the tile) with additional data relevant to double triggers, over the selected time period. Similarly, settings and patient data relevant to auto-PEEP can be shown when the user interacts with the auto-PEEP tiles.

Examples of the types of data presented in the new window (or replaced or augmented tile) include expiratory sensitivity settings, PEEP settings, pressure support settings, peak pressure, plateau pressure, percent support, set tidal volume, expiratory tidal volume, inspiratory time, expiratory time, end-expiratory flow, dynamic resistance (RDYN), dynamic compliance (CDYN), total respiratory rate (Ftot), breath mode, predicted body weight (PBW), and/or other examples of ventilatory and patient data.

In an embodiment, another type of input (tap input, gesture, etc.) on the dashboard opens an asynchrony clock view, described in more detail below. In other examples, the asynchrony dashboard may include an asynchrony clock view in one or more of the tiles or as a replacement for one or more the tiles.

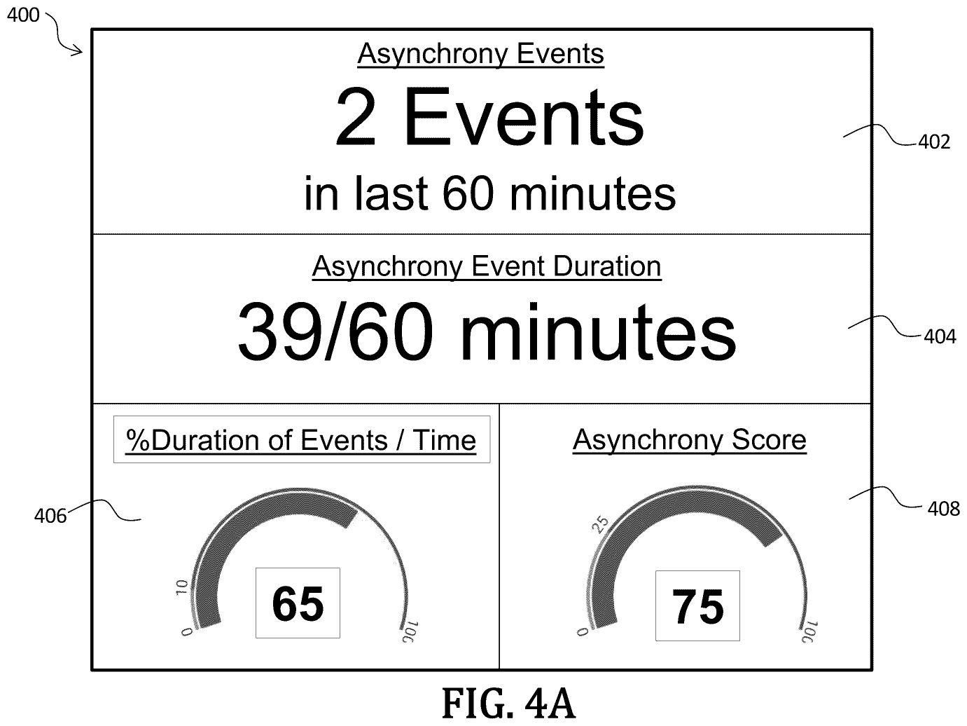

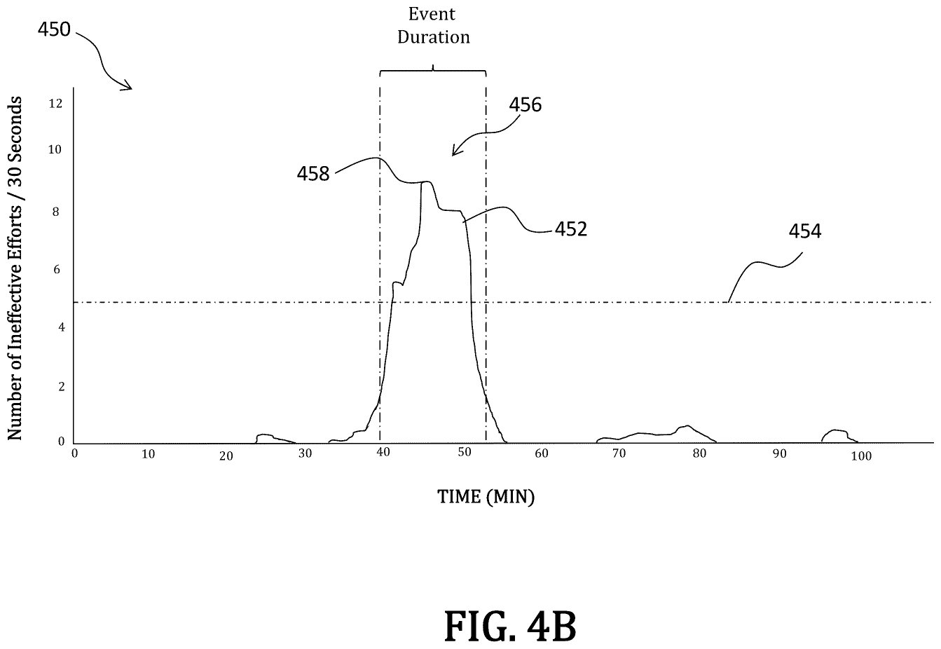

FIG. 4A depicts a view of another asynchrony dashboard 400, or portion thereof, according to an embodiment of the present disclosure. The asynchrony dashboard 400 may be a part of, or incorporated into, one or more of the asynchrony dashboards discussed above. For instance, one or more of the tiles of the asynchrony dashboard 400 may be incorporated into the asynchrony dashboards discussed above. The tiles may be added to the asynchrony dashboards above or replace one or more tiles of the asynchrony dashboards discussed above.

The data indicated or conveyed by the asynchrony dashboard 400 relates to asynchrony events that may be based on a clustering of underlying occurrences and/or a combination of underlying asynchrony occurrences (e.g., ineffective efforts, double triggers) that contribute to asynchrony. For example, the asynchrony events may be based on combinations of the data or occurrences discussed above, such as a combination of ineffective efforts and double triggers that occurred over a particular time period.

The asynchrony events may be based on a weighted combination of the underlying events. For instance, ineffective efforts may be given a higher or smaller weight than a double trigger when determining whether an asynchrony event has occurred. As an example, as discussed above, an ineffective effort threshold was set at 10%, and the double trigger threshold was set at 15%. If either of those thresholds are met, an asynchrony event may be considered to have occurred.

Additionally or alternatively, the asynchrony event may be based on a cluster or repeated asynchrony occurrences. For example, a certain number of ineffective efforts over a period of time may be considered an asynchrony event, which may be referred to as an ineffective-effort-based asynchrony event. One example of an ineffective-effort-based asynchrony event is described in “Clusters of Ineffective Efforts During Mechanical Ventilation: Impact on Outcome,” Intensive Care Med. 2017 February; 43(2):184-191 by K. Vaporidi, et. al. (hereinafter the “Vaporidi Paper”). In that paper, to describe a cluster of ineffective efforts, the concept of an “event” was described. Events of ineffective efforts were defined as periods of time containing more than 30 ineffective efforts in a 3-minute period. Additional discussion of such events is provided below with respect to FIG. 4B. Such events may be considered asynchrony events that are represented in the asynchrony dashboard 400, and those events may be referred to as cluster-based asynchrony events.

The asynchrony events, which are based on clusters or sequences of underlying occurrences (e.g., ineffective efforts), may have both an associated duration and magnitude. The duration may indicate how long the asynchrony event was, which may be indicative of continuing asynchrony issues. The magnitude may indicate the severity of the particular asynchrony event, which may also indicate how far the event diverged from a set threshold. Accordingly, a combination of the following three asynchrony event factors may be particularly useful for a clinician to help immediately assess a situation from a single dashboard: (1) the frequency of the asynchrony events; (2) the duration of the asynchrony events; and (3) the magnitude of asynchrony events.

The asynchrony dashboard 400 includes an event-counter tile 402, an event-duration tile 404, a percent-duration tile 406, and an asynchrony-score tile 408. The event-counter tile 402 indicates a number of asynchrony events that have occurred over a set time duration. In the example depicted, the time duration is set to 60 minutes, and the number of asynchrony events detected over the last 60 minutes was two.

The event-duration tile 404 indicates how much of the set time period was occupied by asynchrony events. For example, each asynchrony event may have an associated duration, which may be long or short. Accordingly, the indication of two events in the event-counter tile 402 may not provide enough information to quickly assess the impact of the asynchrony events. The event-duration tile 404 indicates the remaining duration information to add additional information, which allows for a more immediate assessment. In the depicted example, asynchrony events were occurring over the last 39 of the last 60 minutes. As such, while there were only two asynchrony events over the last 60 minutes, the duration of those events was substantial and occupied over half of the entire hour.

The percent-duration tile 406 provides another indication of the duration of the asynchrony events. The percent-duration tile 406 converts the data from the event-duration tile 404 into a percentage and represents that percentage on a graphical scale. In the present example, the percent of time that an asynchrony event was occurring over the past 60 minutes was 65% (e.g., 39/60 minutes). The percent duration of the asynchrony event(s) may be used effectively as asynchrony index and compared to a threshold. In the example depicted, the duration threshold is set to 10%, which may be indicated in the percent-duration tile 406, such as on the graphical representation. By providing a threshold, the clinician may be able to interpret the impact of the asynchrony events more quickly. In addition, the display of the percent-duration tile 406 may change based on when the percent duration is higher or lower than the threshold. For example, when the percent duration is above the threshold, the format of the text or data in the percent-duration tile 406 may be changed (e.g., different color (red), highlighted, bolded).

The asynchrony-score tile 408 indicates an asynchrony score, which may be used as an asynchrony index. The asynchrony score may be a score that is based the frequency, magnitude, and/or duration of the asynchrony events occurring over the set time period. For instance, the asynchrony score may be based on at least two of: the frequency, magnitude, and/or duration of the asynchrony events. The asynchrony score may be a weighted combination of those factors. As a few examples, the magnitude of the events may be weighted more heavily than the duration, or the total duration may be weighted more heavily than the frequency. In the example depicted, the asynchrony score is a number between 0-100, but other scales and representations of the asynchrony score are possible.

The asynchrony-score tile 408 may also include a graphical scale similar to the percent-duration tile 406. In the example depicted, the scale ranges from 0-100 and also indicates a threshold of 25. Accordingly, in this example, an asynchrony score below the threshold of 25 may be considered less concerning, and by displaying the threshold and the current asynchrony score in the same tile, the clinician is able to quickly assess the severity of the asynchrony score. In the example depicted, the asynchrony score is 75, which significantly exceeds the threshold of 25. When the asynchrony score exceeds the threshold, the format of the text or data in the asynchrony-score tile 408 may be changed (e.g., different color (red), highlighted, bolded).

FIG. 4B depicts a plot 450 for identifying cluster-based asynchrony events according to an embodiment of the present disclosure. The plot 450 indicates one manner in which cluster-based asynchrony events may be identified, which is similar to the manner in which the events of the Vaporidi Paper are identified.

To detect the cluster-based asynchrony event, an ineffective effort signal (an IE signal) may be generated that represents a number of ineffective efforts that occur over a time period. For example, the IE signal may be a number of ineffective efforts that occur in each 30 second interval. This IE signal may be smoothed and/or generated by using a moving average window. An example of such an IE signal is depicted as IE signal 452 in plot 450.

In the current example, the y-axis of the plot 450 represents the number of ineffective efforts that have occurred in each 30 second interval (or the particular scale used for the IE signal 452). The x-axis represents time and has a scale of minutes in this example (other time scales are possible). An event threshold 454 is represented on the plot 450. The threshold 454 defines whether an event is occurring or not. For instance, in the present example, the event threshold is 30 ineffective efforts in a three-minute period. Within the scale presented on the plot 450, the threshold 454 may be equivalent to 5 ineffective efforts per 30 second interval. Accordingly, when the IE signal 452 exceeds a value of 5 ineffective efforts per 30 second interval, the IE signal 452 has crossed the threshold 454. When the IE signal 452 crosses the threshold 454 an event is determined to have occurred. An example event 456 is shown in plot 450. The event 456 may not be a singular point in time but rather may have a duration as shown on the plot 450. The event also has a maximum value 458 of the IE signal 452 during the event. The maximum value 458 may be representative of the magnitude of the event.

The duration of the event may be the time from when the IE signal 452 is at 20% of the maximum value 458 (prior to the occurrence of the maximum value 458) to when the IE signal 452 is again at 20% of the maximum value 458 (after the occurrence of the maximum value). In other examples, the duration of the event may be the time from when the IE signal 452 crosses the threshold 454 at the beginning of the event and when the IE signal 452 crosses the threshold 454 at the end of the event.

The duration and magnitude of the event may be represented by an area under the IE signal 452 (e.g., integral of the IE signal 452) for the duration of the event. For example, the total number of ineffective efforts that occurred during the event may be determined, which may be considered the “power” of the event. The power of the event (e.g., the number of ineffective efforts during the event) may be used as a basis or factor for the asynchrony score discussed above regarding the asynchrony-score tile 408. For instance, the power may be scaled or normalized to generate an asynchrony score. The asynchrony score may also be based on other factors, such as duration, magnitude, and/or other underlying events or data.

While the above plot and discussion has been specific to the underlying ineffective efforts as contributing the detection of cluster-based asynchrony events, similar clustering and analysis may be performed for different underlying criteria, such as double triggers. Different thresholds may also be used for different types of criteria (e.g., different types of asynchronies). In such examples, the IE signal 452 may generally be referred to as an asynchrony signal that represents a number of underlying occurrences (e.g., double triggers, ineffective efforts) that are happening per time frame (e.g., 30 seconds, 1 minute, 2 minutes). The asynchrony events detected from asynchrony signals of different asynchrony types may be combined (e.g., in a weighted combination) to generate the asynchrony event data set forth in the dashboard 400. As an example, events detected from the IE signal 452 and events detected from a double-trigger signal may be combined to form the asynchrony-event data and/or the asynchrony score in dashboard 400. Accordingly, the duration, magnitude, power, and/or frequency of different types of asynchronies may taken into account.

The plot 450, or a version thereof, may also be included in a tile in an asynchrony dashboard, such as the asynchrony dashboard 400 discussed above. Accordingly, a clinician may be able to discern information about the asynchrony events, such as magnitude, duration, and frequency, directly from the plot 450 in the asynchrony dashboard 400. The time scale or duration of the plot may be set to the set time duration for the asynchrony dashboard 400.

In addition to displaying asynchrony data based on the detection of such events, the ventilator may also generate alarms based on the duration and/or magnitude of the events. For example, if the duration and/or magnitude exceeds a corresponding threshold, an alarm may be activated. Additionally or alternatively, if the power of a particular event exceeds a threshold, an alarm may be activated.

Asynchrony Clock

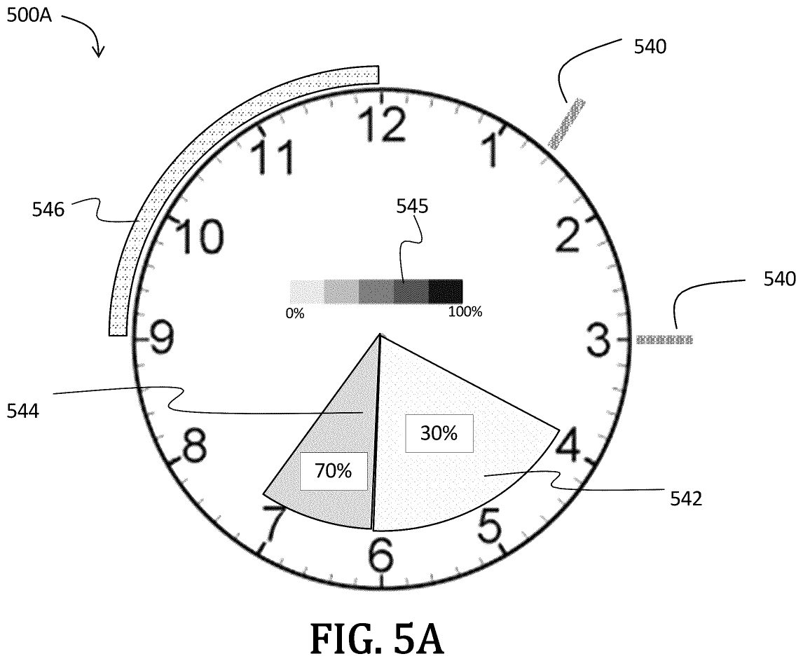

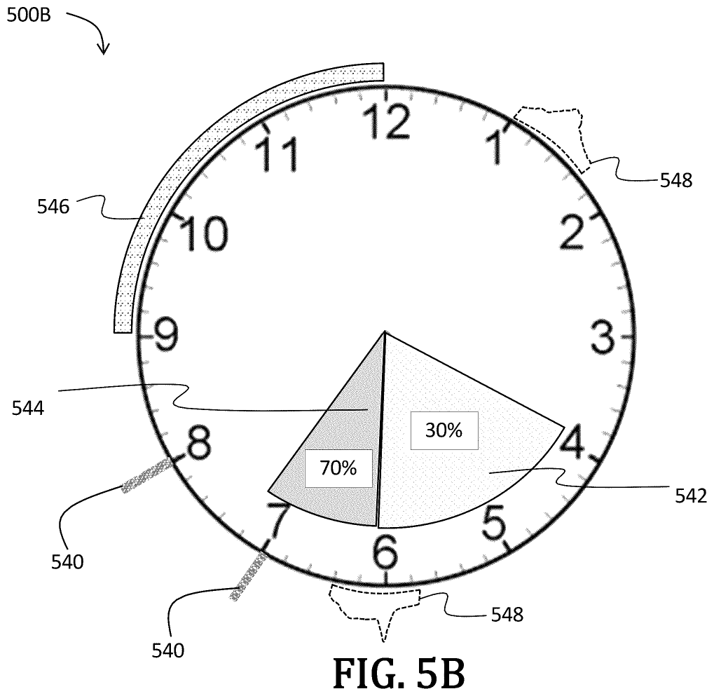

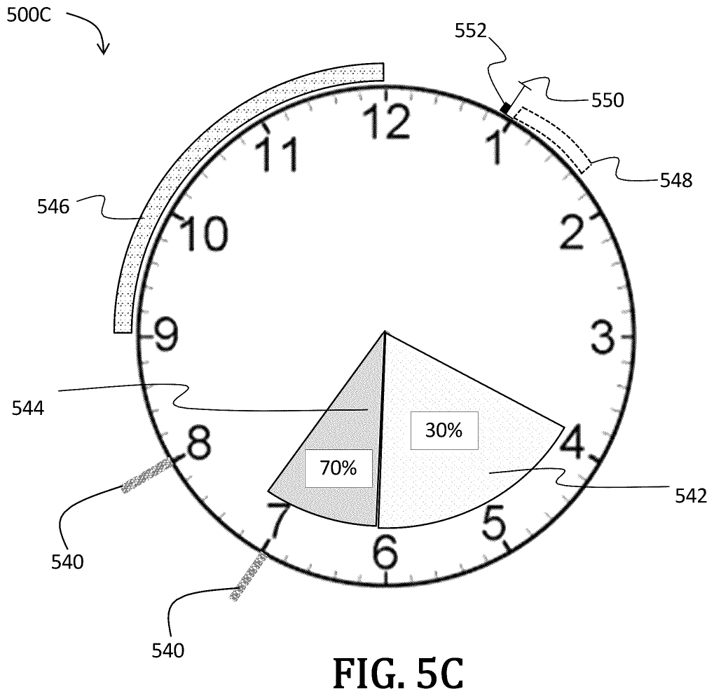

FIG. 5A shows another view of asynchrony data, presented in the form of an asynchrony clock 500A. The clock 500A is a 12-hour clock, showing noon to midnight. Tick markers 540 on the periphery of the clock indicate an event, such as a ventilator settings change, a medication dosage, or others listed below. Shaded regions or areas 542 and 544 indicate the presence of asynchrony during the shaded time. Area 542 is shaded a lighter color or texture, indicating a less severe asynchrony is present, and area 544 is shaded a darker color or texture, indicating a more severe asynchrony. For example, in clock 500A, during the time shaded by area 542, the patient experienced the particular type of asynchrony in 30% of breaths. During the time shaded by area 544, the patient experienced the asynchrony in 70% of breaths. In an embodiment, the amount of shading or texturing is proportional to the severity of the asynchrony (such as the shading getting darker in proportion to the percentage of asynchronous breaths increasing). That formatting may change within a single area 544 over time to indicate changes in severity. In some examples, the entire area within the asynchrony clock 500A may be filled with area indicators (such as area 542 and 544). The format (e.g., color) the area may then change at different points in time to represent the severity of the asynchrony.

In examples where color or similar formatting of the indicators or areas is used to convey additional information, a scale or legend 545 may be displayed inside or outside of the asynchrony clock 500A. The legend 545 indicates the values to which the colors or formats correspond. For instance, a clear or light color may correspond to a low percentage or level, and a darker color may indicate a higher percentage or level.

Additional graphical indicators, icons, and markers can show additional data, such as arc 546 which indicates the patient's awake/sleep status (the arc indicating times when the patient is sleeping or sedated). The event indicated by the tick marker 540, arc 546, or other markers can represent one or more of ventilator operating data (alarm status, settings changes, breath mode changes), medication delivery (doses delivered to a patient), sedation level, meals, suction procedures, caretaker shift changes, and other relevant data. These markers can be pushed to the clock automatically (such as when the ventilator registers a settings change or alarm status change) or manually (such as a clinician adding data to the dashboard or other interface to show that suction, medication, or other events took place).

For example, a sedation level of the patient may be determined from a sensor, such as a depth camera sensor, and/or from a bispectral index (BIS) value. The sedation level may be displayed as an arc 546 or shaded area 544 to convey the duration of the sedation and/or the level of sedation. In some examples, the indicator for the sedation level may be displayed only for levels of sedation that are above a particular threshold. The same or similar indicators may be presented or displayed for sleep rather than sedation.

Indicators, such as tick markers 540, may also be automatically displayed by the ventilator when a ventilation settings change occurs. Thus, the clinician can readily see when ventilation settings changes were made relative to the asynchrony data. The indicator for the ventilation settings change may also be formatted to indicate different types of ventilation settings. For example, a marker 540 for a change in ventilation mode may be indicated in one format (e.g., color, size, shape), and a marker 540 for a change in a parameter or option for a particular mode (e.g., less major change) may be indicated in a different format.

As another example, other types of events may be manually added to the asynchrony clock 500A by user. For instance, by selecting a portion or location on the clock, a user interface to add details for an event may be displayed. User input into the user interface may then be received and processed to generate a corresponding element or marker 540 on the clock based on the event details received in the user interface. As example, an event type (e.g., medication delivery vs. meals) may be added and distinguished by the formatting the corresponding marker 540. The manual addition of markers 540 may be particularly useful for events that are not detectable by sensors in communication with the ventilator. Where appropriate, additional details about the event may also be provided, such as the duration of the event. An indicator is then generated and displayed on the clock at the selected location, and the indicator format may be based on the event type and/or the additional information received about the event.

Accordingly, the asynchrony clock 500A provides a single interface that is able to represent both asynchrony events and non-asynchrony events. Indeed, in some examples, the events represented on the asynchrony clock are not ventilatory events (e.g., sleep, meals, medication). The single interface view provides a readily assessable tool for a clinician to determine potential cause-and-effect relationships between asynchrony events and the non-asynchrony events. For example, the impact of a meal or sleep on asynchronies may become readily apparent from the asynchrony clock 500A. Similarly, the impact of a ventilator-settings change on asynchrony may also become readily apparent from the asynchrony clock 500A.

While the asynchrony clock 500A is depicted as a 12-hour clock, in other embodiments, the asynchrony clock 500A can span other time periods, such as a 24-hour clock to show one complete day, an 8-hour clock to show one complete shift, or other relevant time periods. For instance, the clock may also be set to a single hour or other duration to show a more granular view of the asynchrony data. Displaying asynchrony information on a clock face enables asynchrony events to be more readily understood in context by a clinician.

FIG. 5B shows another example of an asynchrony clock 500B. The asynchrony clock 500B depicted in FIG. 5B includes the same type of indicators as the asynchrony clock 500A depicted in FIG. 5A. The asynchrony clock 500B also includes a cluster-based asynchrony event indicator 548. The cluster-based asynchrony event indicator 548 indicates information about detected cluster-based asynchrony events, such as the event discussed above with respect to FIG. 4B. The cluster-based asynchrony event indicator 548 may indicate the occurrence of the asynchrony event as well as additional details about the asynchrony event. For instance, in the example depicted, the cluster-based asynchrony event indicator 548 starts at the beginning of the asynchrony event and ends at the end of the asynchrony event. Because of the asynchrony clock 500B, the cluster-based asynchrony event indicator 548 indicates not only the duration of the corresponding asynchrony event but also the time of day at which the cluster-based asynchrony event indicator 548 occurred.

The cluster-based asynchrony event indicator 548 in asynchrony clock 500B also includes an outer boundary that indicates the magnitude of the event over the duration of the event. For instance, the outer boundary of the cluster-based asynchrony event indicator 548 may have a similar shape to the underlying signal that is monitored for determining the event, such as the IE signal 452 in FIG. 4B. By showing the boundary based on, or shaped similarly to, the IE signal 452, the clinician is able to further assess the magnitude of the associated asynchrony event.

A different cluster-based asynchrony event indicator 548 may be displayed on the asynchrony clock 500B for each asynchrony event that is detected. The characteristics of each cluster-based asynchrony event indicator 548 may be based on the particular asynchrony event. For instance, different cluster-based asynchrony event indicators 548 may have different sizes (indicating different durations) and/or differently shaped outer boundaries (corresponding to the different underlying signals of the events). Other forms of showing magnitude of the cluster-based asynchrony event may also be used alternatively to the boundary of the cluster-based asynchrony event indicator 548 being shaped similarly to the IE signal (or other asynchrony signal).

FIG. 5C shows another example of an asynchrony clock 500C. The asynchrony clock 500C is substantially the same as asynchrony clock 500B with the exception that the cluster-based asynchrony event indicator 548 in asynchrony clock 500C has a different format from the cluster-based asynchrony event indicator 548 in asynchrony clock 500B. In asynchrony clock 500C, the cluster-based asynchrony event indicator 548 does not include an outer boundary that is shaped to match the underlying asynchrony signal. Rather, the height of the cluster-based asynchrony event indicator 548 provides information about the magnitude of the event. The height may be based on the peak magnitude of the asynchrony signal (e.g., peak IE signal value) over the asynchrony event, the average of the asynchrony signal over the asynchrony event, and/or the power of the asynchrony event (e.g., the number of ineffective efforts or double triggers during the event).

A scale 550 and threshold indicator 552 may also be included in the cluster-based asynchrony event indicator 548. For example, the scale 550 indicates the maximum possible height for the cluster-based asynchrony event indicator 548 to provide a frame of reference. The threshold indicator 552 indicates the asynchrony threshold, similar to the thresholds shown in the tiles discussed above, such as the percent-duration tile 406 or the asynchrony-score tile 408.

In yet other examples of a cluster-based asynchrony event indicator 548, the color and/or fill of the cluster-based asynchrony event indicator 548 may change based on the magnitude or power of the corresponding event. For instance, the color may change throughout the cluster-based asynchrony event indicator 548 to show a time change in the magnitude of the asynchrony signal. In other examples, the entire cluster-based asynchrony event indicator 548 may be one color that is based on the power or average signal during the event. Other format changes are also possible to indicate the magnitude or severity of the corresponding event.

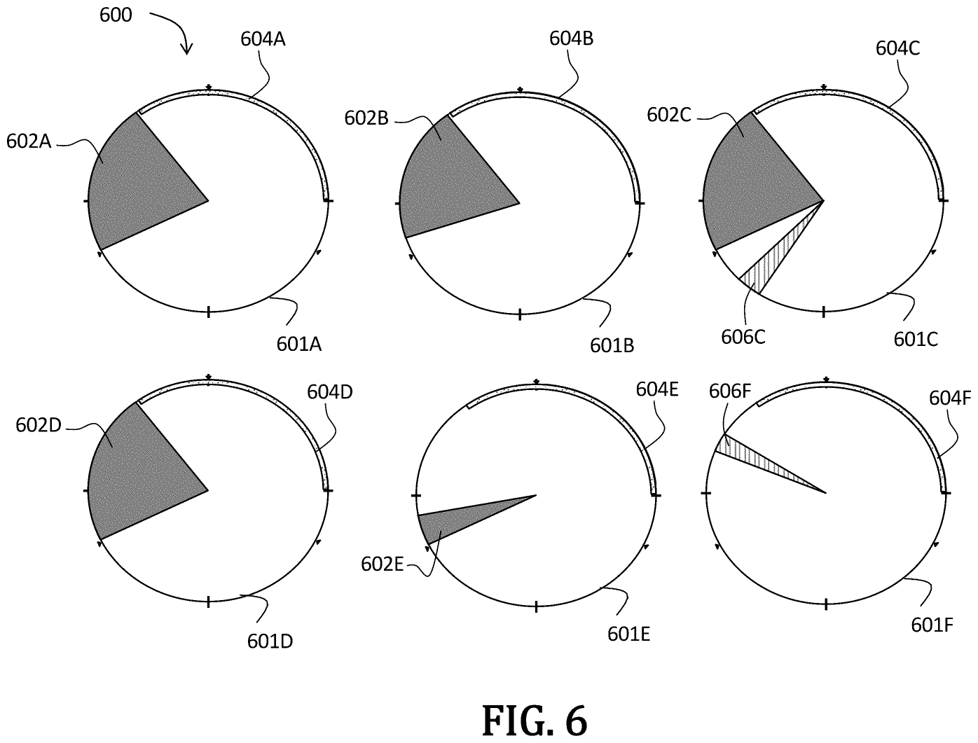

FIG. 6 shows a dashboard view 600 of multiple asynchrony clocks 601A-F over a series of periods. This dashboard view 600 of clocks enables a user to see trends across the same time each day, such as identifying that asynchrony repeats after a meal, after a medication dosage, before or after sleep, when a ventilator mode was changed, etc. For example, the clocks 601A-F may all be 24-hour clocks that each represent a different day. Accordingly, in the present example, six different days may be quickly assessed from a single display.

For example, a first indicator 602A-E having a first indicator type is displayed in 5 of the 6 clocks. The first indicator 602A-E may represent a particular type of asynchrony (e.g., missed breath or double trigger). Thus, a quick assessment of that first indicator type can be across multiple days to see difference in durations and the lack of that indicator type on the 6^{th} clock 601F. In an example, where the first indicator 602A-E indicates missed-breath asynchrony, a gradual reduction across the days in the missed-breath asynchrony can be seen from the series of asynchrony clocks 601A-F until the missed-breath asynchrony is no longer present on the last day.

Similarly, a second indicator 604A-F, corresponding to a second indicator type, is shown on each of the on each of the clocks 601A-F. This second indicator 604A-F may, for example, be indicative of sleep time and duration for the patient.

In contrast, a third indicator 606C,F, corresponding to a third indicator type, is shown only on two of the clocks 601C,F. The third indicator 606C,F may indicate a different type of asynchrony from the first indicator 602A-E. Continuing with the example above where the first indicator 602A-E represents a missed-breath asynchrony, the third indicator 606C,F may indicate a double-trigger asynchrony. While not explicitly depicted in FIG. 6, the asynchrony clocks 601A-F may include any of the types of indicators or markers that are discussed as being included with the asynchrony clocks of FIGS. 5A-5C.

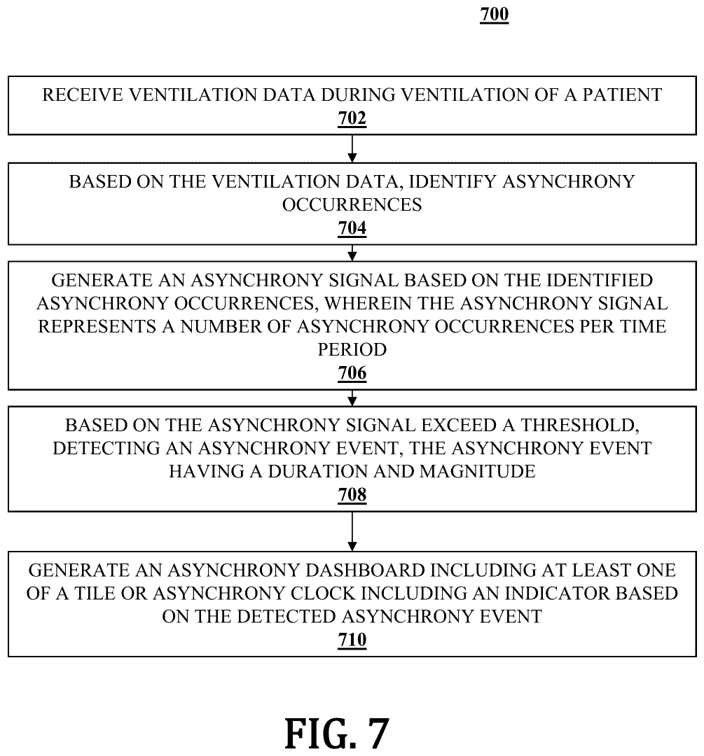

FIG. 7 depicts an example method 700 for generating an asynchrony dashboard. The example method 700 may be performed by a ventilator and/or a remote device. For example, the ventilator and/or remote device may include a processor and memory. The memory stores instructions that, when executed by the processor, causes the ventilator and/or remote device to perform the operations of method 700.

At operation 702, ventilation data is received during ventilation of a patient. For instance, the ventilator may deliver ventilation (e.g., breathing gases) to the patient. While the ventilation is being delivered, sensors of the ventilator measure or transduce ventilation data, such as pressures and flows of the breathing gases flowing through the breathing circuit. The captured ventilation data from the sensors may also be used to calculate or generate additional ventilation data, such as volumes, effort signals, or other representative signals about the ventilation.

At operation 704, asynchrony occurrences are identified based on the ventilation data received at operation 702. The asynchrony occurrences may be each instance of an ineffective effort, double trigger, premature cycling, delayed cycling, etc. The asynchrony occurrences may be detected as discussed herein and in the references that are incorporated herein.

At operation 706, an asynchrony signal is generated based on the identified asynchrony occurrences. The asynchrony signal represents a number of asynchrony occurrences per time frame or period. One example of an asynchrony signal is the IE signal 452 discussed above. The IE signal 452 represents a number of ineffective efforts that occur over a time period. For example, the IE signal 452 may be a number of ineffective efforts that occur in each 30 second interval. The asynchrony signal generated in operation 706 may be for different types of asynchrony occurrences other than ineffective efforts and/or the time period may be different than 30 seconds. The asynchrony signal may be smoothed and/or averaged signal based on detected asynchrony occurrences.

At operation 708, an asynchrony event is detected based on the asynchrony signal exceeding an asynchrony threshold. For example, the asynchrony threshold may be based on a number of the asynchrony occurrences that occur over a time period, such as 30 asynchrony occurrences in a three-minute period (or 5 asynchrony occurrences in 30 seconds). When the asynchrony signal crosses the asynchrony threshold, the asynchrony event is detected, which may be referred to as a cluster-based asynchrony event. The asynchrony event has a duration and magnitude, which may be calculated as discussed above. A power or integral of the asynchrony signal during the asynchrony event may also be generated. The power may represent the number of asynchrony occurrences that occurred during the asynchrony event.

At operation 710, an asynchrony dashboard is generated that includes at least one of a tile or asynchrony clock including an indicator based on the detected asynchrony event. For instance, the tiles or asynchrony clocks discussed above may be generated and include corresponding data based on the detected asynchrony event. The method 700 may repeat and additional asynchrony events may be detected. As the additional asynchrony events are detected, the asynchrony dashboard may be updated.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A system for generating a patient-ventilator asynchrony dashboard comprising:
a processor receiving ventilation data from a medical ventilator and outputting a data dashboard on a display screen;
wherein the data dashboard displays a detection of first and second different types of patient-ventilator asynchrony events in the ventilation data; and
wherein the data dashboard comprises time-scaled asynchrony metrics organized into tiles for each of the first and second asynchrony events.

2. The system of claim 1, wherein the first type of patient-ventilator asynchrony events includes ineffective efforts.

3. The system of claim 2, wherein the second type of patient-ventilator asynchrony events includes double triggers.

4. The system of claim 1, wherein at least one of the tiles indicates a number of minutes out a set time period that the first type of patient-ventilator asynchrony events exceeded a threshold.

5. The system of claim 1, wherein at least one of the tiles indicates percentage of time out a set time period that the first type of patient-ventilator asynchrony events exceeded a threshold.

6. The system of claim 1, wherein the data dashboard further comprises an asynchrony clock including at least one indicator representing an asynchrony.

7. The system of claim 6, wherein the at least one indicator indicates a time, duration, and severity of the asynchrony.

8. A ventilator-implemented method for generating an asynchrony dashboard, the method comprising:
receiving ventilation data during ventilation of a patient;
based on the ventilation data, identifying asynchrony occurrences;
generating an asynchrony signal based on the identified asynchrony occurrences, wherein the asynchrony signal represents a number of asynchrony occurrences per time frame;
based on the asynchrony signal exceeding a threshold, detecting an asynchrony event, the asynchrony event having a duration and magnitude; and
generating an asynchrony dashboard including at least one of tile or asynchrony clock including an indicator based on the detected asynchrony event, wherein the indicator indicates at least one of the duration or the magnitude of the detected asynchrony event.

9. The ventilator-implemented method of claim 8, wherein the asynchrony signal is an ineffective effort signal.

10. The ventilator-implemented method of claim 8, wherein the asynchrony dashboard includes an asynchrony score based on at least one of duration or the magnitude of the asynchrony event.

11. The ventilator-implemented method of claim 10, wherein the asynchrony score is based on a power of the asynchrony event.

12. The ventilator-implemented method of claim 11, wherein the power indicates a number of asynchrony occurrences that occurred during the asynchrony event.

13. The ventilator-implemented method of claim 10, wherein the dashboard includes the asynchrony clock.

14. The ventilator-implemented method of claim 13, wherein the indicator indicates the magnitude, duration, and time of the asynchrony event.

15. The ventilator-implemented method of claim 8, wherein the asynchrony clock further includes at least one additional indicator corresponding to a manually entered event.
